(19) United States
(12) Patent Application Publication
(10) Pub. No.: US 2025/0281708 A1
(43) Pub. Date: Sep. 11, 2025

(54) FLUID DRAINING RESPIRATORY MASK

(71) Applicant: Mitchell Ishizu, Anaheim, CA (US)

(72) Inventor: Mitchell Ishizu, Anaheim, CA (US)

(21) Appl. No.: 18/601,770

(22) Filed: Mar. 11, 2024

(51) Int. Cl. A61M 16/06; A61M 16/08

(52) U.S. Cl. CPC A61M 16/0677; A61M 16/0644; A61M 16/085; A61M 2205/3334

(57) ABSTRACT

A respiratory mask for facilitating a patient's breathing via a pressure generation device includes a body that defines a breathing cavity that can be positioned receive a nose and a mouth of a patient. A positive pressure tube and a negative pressure tube are in fluid communication with the breathing cavity via separate apertures. The positive pressure tube may couple to a pressure generation device which causes a gas to flow into the breathing cavity to assist the patient in breathing. The negative pressure tube may be coupled to a suction device which is operable to collect fluid from the breathing cavity such as vomit, spit, or the like.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to respiratory masks and more particularly pertains to a new respiratory mask for facilitating a patient's breathing via a pressure generation device while draining bodily fluids from the mask.

### (2) Description of Related Art Including Information Disclosed Under 37 CFR 1.97 and 1.98

The prior art discloses myriad respiratory masks for use with pressure generation devices such as bilevel positive airway pressure (BiPAP) machines or like devices. U.S. Pat. No. 8,844,530 describes such a device which has a nasal interface and a separate mouth interface such that a suction device may remove mucus from a patient's mouth via the mouth interface while a pressure generation device delivers pressurized air to the patient's lungs via their nose. However, patients who are unconscious or otherwise unable to keep their mouth in fluid communication with the mouth interface would not be able to use such a device in the event that they vomit or produce other bodily fluids from the mouth that need to be drained. The prior art fails to describe such a device which places tubes in communication with an entire breathing cavity of a mask such that vomit and other fluids may be removed from the breathing cavity simultaneously with a pressure generation device assisting the patient with breathing.

### BRIEF SUMMARY OF THE INVENTION

An embodiment of the disclosure meets the needs presented above by generally comprising a body which defines a breathing cavity and has a rim that defines an opening to the breathing cavity. The body is shaped and configured to receive a nose and a mouth of a patient through the opening into the breathing cavity. The body also defines a positive pressure aperture and a negative pressure aperture which extend into the breathing cavity opposite the opening. The negative pressure aperture is positioned between the positive pressure aperture and a bottom end of the body.

A positive pressure tube has a first end which is coupled to the body and is in fluid communication with the positive pressure aperture. A second end of the positive pressure tube is configured to be in fluid communication with a positive pressure source to receive a flow of gas into the breathing cavity via the positive pressure tube. A negative pressure tube has a first end which is coupled to the body and is in fluid communication with the negative pressure aperture. A second end of the negative pressure tube is configured to be in fluid communication with a negative pressure source to facilitate fluid flowing out of the breathing cavity via the negative pressure tube.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a front perspective view of a respiratory mask according to an embodiment of the disclosure.

FIG. 2 is another front perspective view of an embodiment of the disclosure.

FIG. 3 is a front view of an embodiment of the disclosure.

FIG. 4 is a rear view of an embodiment of the disclosure.

FIG. 5 is a side view of an embodiment of the disclosure.

FIG. 6 is a cross-section view of an embodiment of the disclosure taken from Line 6-6 in FIG. 3.

FIG. 7 is an in-use view of an embodiment of the disclosure.

### DETAILED DESCRIPTION OF THE INVENTION

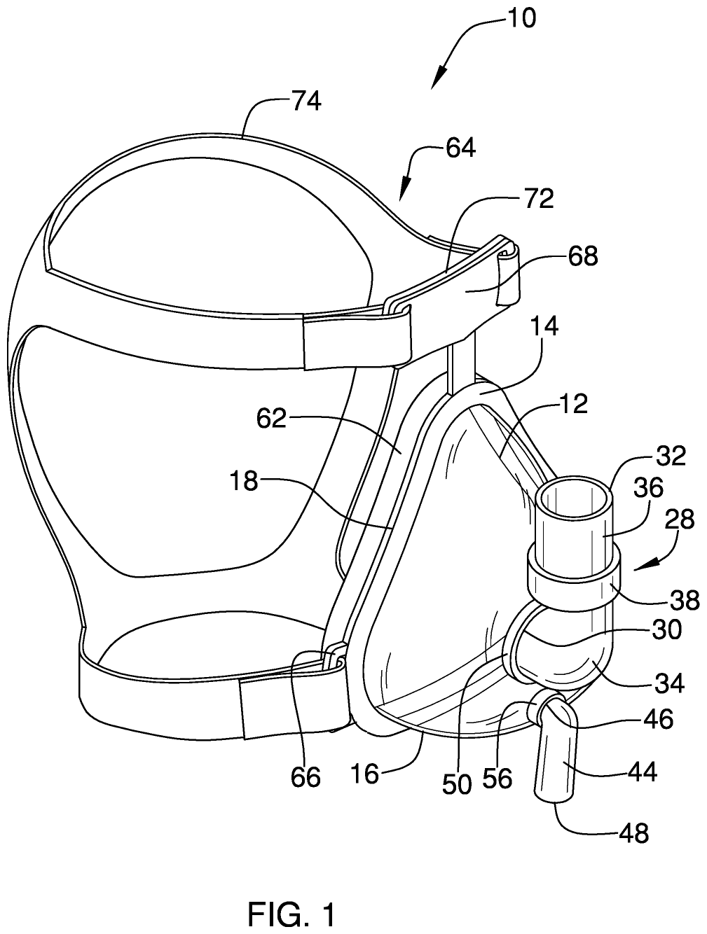

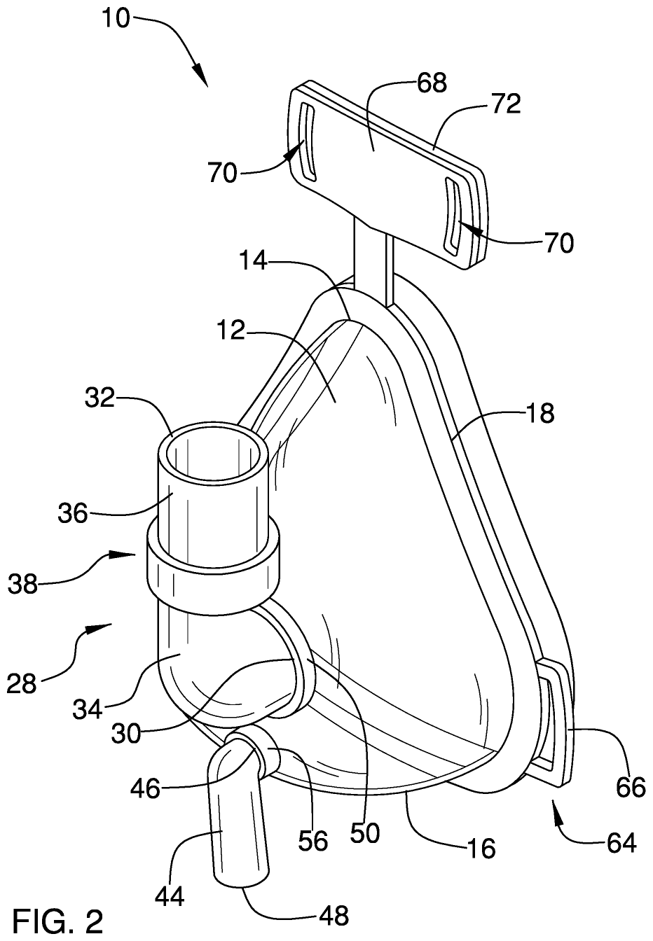

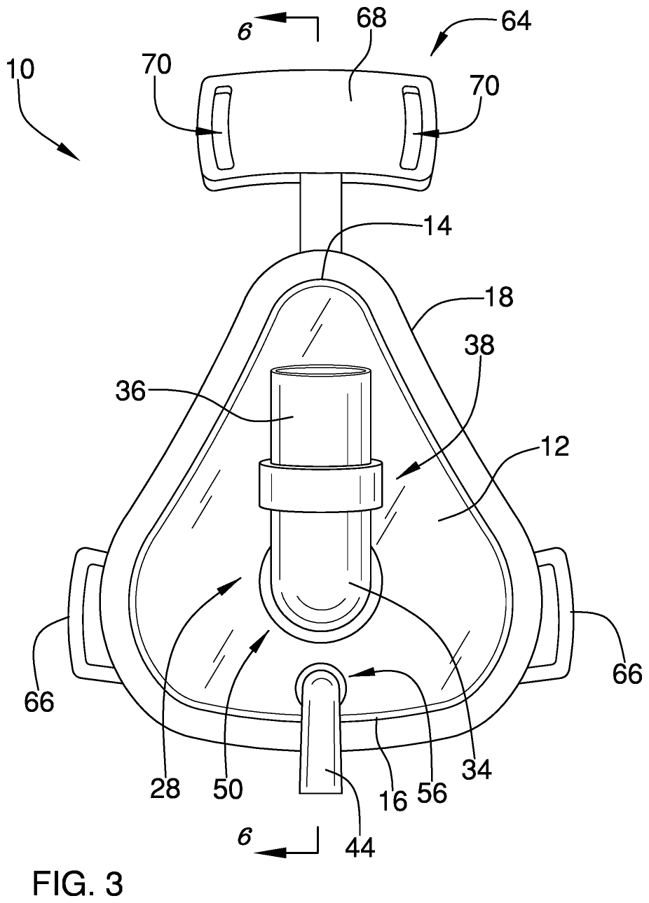

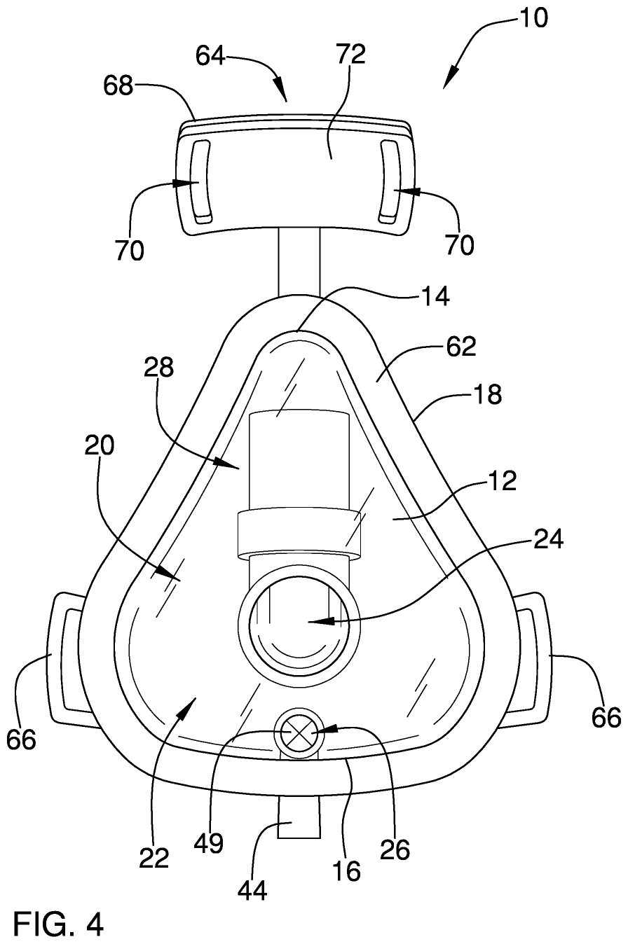

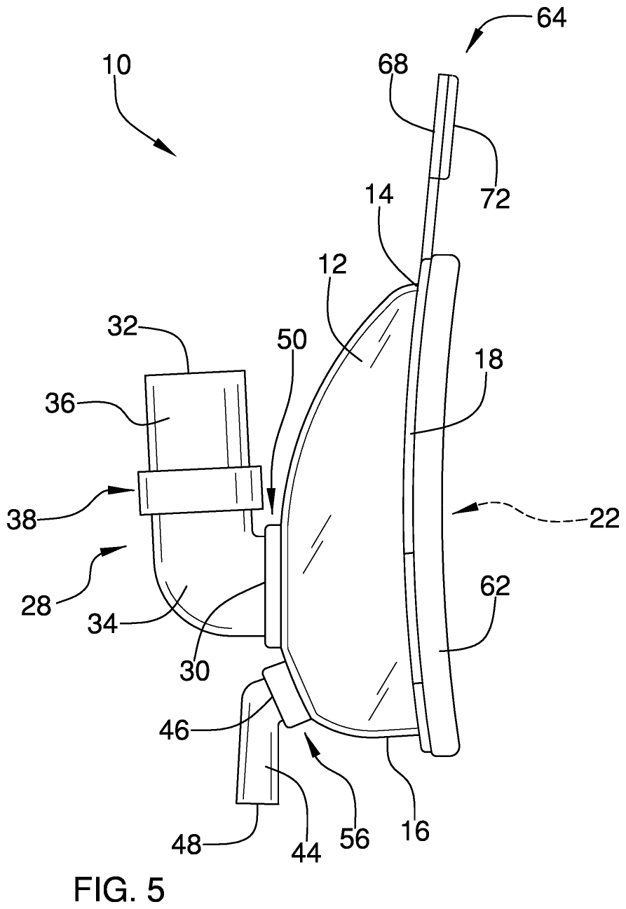

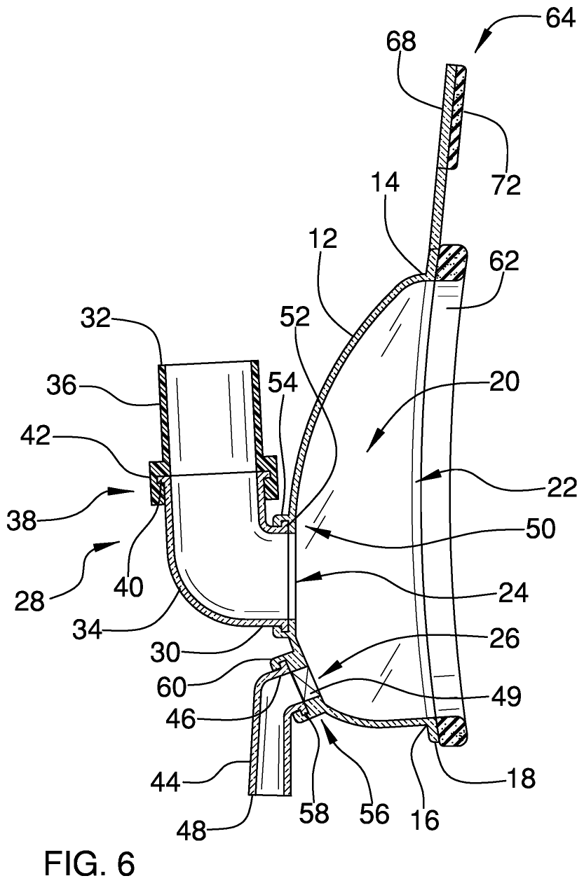

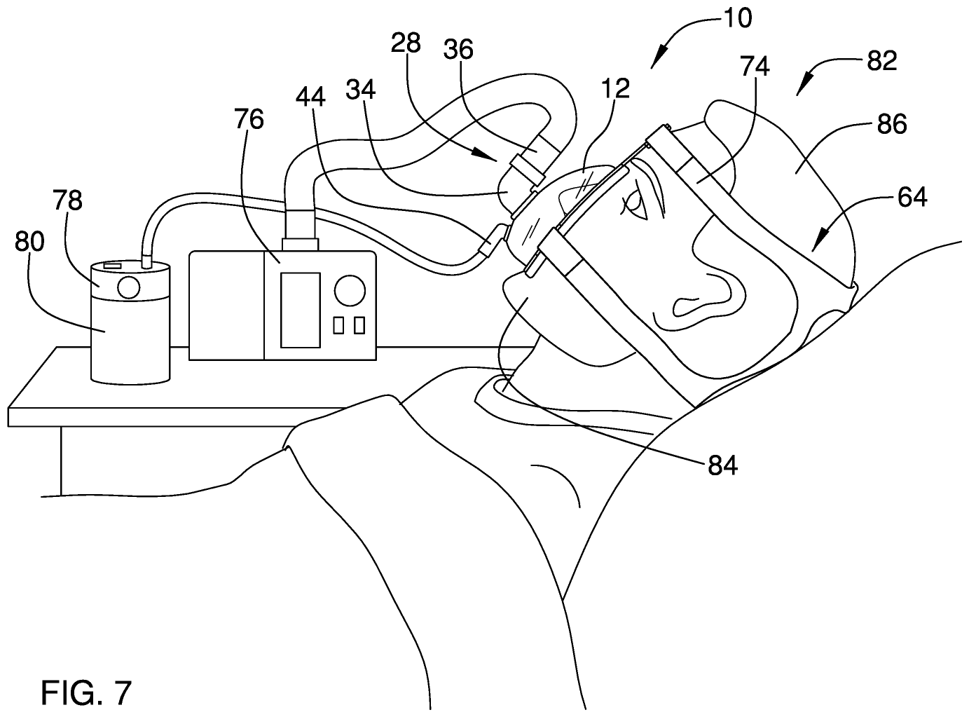

With reference now to the drawings, and in particular to FIGS. 1 through 7 thereof, a new respiratory mask embodying the principles and concepts of an embodiment of the disclosure and generally designated by the reference numeral 10 will be described.

As best illustrated in FIGS. 1 through 7, the respiratory mask 10 generally comprises a body 12 which defines a breathing cavity 20 and has a rim 18 which defines an opening 22 to the breathing cavity 20. The body 12 is shaped and configured to receive a nose and a mouth of a patient 82 through the opening 22 into the breathing cavity 20. The body 12 defines a positive pressure aperture 24 and a negative pressure aperture 26 which extend into the breathing cavity 20 opposite the opening 22. The negative pressure aperture 26 is positioned between the positive pressure aperture 24 and a bottom end 16 of the body 12.

A positive pressure tube 28 has a first end 30 which is coupled to the body 12 and is in fluid communication with the positive pressure aperture 24. A negative pressure tube 44 also has a first end 46 which is coupled to the body 12 and which is in fluid communication with the negative pressure aperture 26. The positive pressure tube 28 connects at a second end 32 to a pressure generation device 76 which is configured to cause gas flow away from the pressure generation device 76 via positive pressure. The pressure generation device 76 may be a bilevel positive airway pressure (BiPAP) machine, a continuous positive airway pressure (CPAP) machine, or the like. A suction device 78 is coupled to a second end 48 of the negative pressure tube 44. The suction device 78 is operable to apply negative pressure to the negative pressure tube 44 in order to draw fluid such as vomit, spit, or other bodily fluids from the breathing cavity 20 of the body 12, through the negative pressure tube 44, and into a chamber 80 of the suction device 78. The suction device 78 may be operated intermittently, continuously, or on demand as is desirable. A one-way valve 49 is coupled to the body 12 and positioned in the negative pressure aperture 26. The one-way valve 49 is configured to allow fluid to pass only from the breathing cavity 20 of the body 12 into the negative pressure tube 44.

The positive pressure tube 28 includes a first tube segment 34 and a second tube segment 36. The first tube segment 34 extends between the first end 30 of the positive pressure tube 28 and the second tube segment 36, and the second tube segment 36 extends between the first tube segment 34 and the second end 32 of the positive pressure tube 28. The first tube segment 34 has an L-shape so that further tubing connected to the positive pressure tube 28 may be draped across the patient 82 or a nearby surface 84 such as a bed. The positive pressure tube 28 includes a segment swivel connector 38 which is configured to allow the second tube segment 36 to rotate with respect to the first tube segment 34. The segment swivel connector 38 includes a segment annular flange 40 attached to the second tube segment 36 and a segment flange receiver 42 attached to the first tube segment 34. The segment annular flange 40 is seated in the segment flange receiver 42. The negative pressure tube 44 also has an L-shape to allow tubing to drape as described.

The positive pressure tube 28 and the negative pressure tube 44 are permitted to swivel with respect to the body 12 by a positive pressure swivel connector 50 and a negative pressure swivel connector 56 respectively. The positive pressure swivel connector 50 comprises a positive pressure flange 52 attached to the positive pressure tube 28 and a positive pressure receiver 54 attached to the body 12, wherein the positive pressure flange 52 is seated in a groove defined by the positive pressure receiver 54. The negative pressure swivel connector 56 comprises a negative pressure flange 58 attached to the negative pressure tube 44 and a negative pressure receiver 60 attached to the body 12, wherein the negative pressure flange 58 is seated in a groove defined by the negative pressure receiver 60.

A sealing member 62 is coupled to the rim 18 of the body 12 and comprises a malleable material such that the sealing member 62 is configured to form a seal between the body 12 and a face 84 of the patient 82. The sealing member 62 may comprise a rubber, a silicone, or the like. An attachment harness 64 is attached to the body 12 to attach the body 12 to the head 86 of the patient 82. The attachment harness 64 comprises a pair of loops 66, a forehead 86 brace 68, and a webbing 74. The loops 66 are attached to the body 12 and are laterally spaced from each other. The forehead 86 brace 68 is mounted to a top end 14 of the body 12 and is configured to abut a forehead 86 of the patient 82. The forehead 86 brace 68 includes a cushion 72 to avoid scratching or other irritation to the patient's 82 skin. The webbing 74 is attached to each of the loops 66 and to the forehead 86 brace 68 and is shaped to extend behind the head 86 of the patient 82. The webbing 74 has strap segments which extend through the loops 66 and through slots 70 in the forehead 86 brace 68. The webbing 74 may comprise an elastic material such as rubber, nylon, neoprene, or the like to stretch around the head 86 and pull the body 12 toward the patient's 82 face 84. The webbing 74 also may comprise adjustable straps to close the body 12 against the patient's 82 face 84.

In use, the patient 82 wears the respiratory mask 10 and is assisted in breathing via the positive pressure exerted on the breathing cavity 20 of the body 12 by the pressure generation device 76. In the event the patient 82 vomits or spits up while wearing the respiratory mask 10, the suction device 78 applies negative pressure to draw such fluids out of the breathing cavity 20 and deposit them in the chamber 80. The suction device 78 may be made to operate continually in conjunction with the pressure generation device 76 such that the patient 82 may continue to be assisted to breath, but any fluids that are deposited into the breathing cavity 20 are automatically removed. For example, the suction device 78 may continuously apply negative pressure at a low enough magnitude that the pressure generation device 76 still causes a positive pressure to the patient's 82 lungs but a high enough magnitude to draw fluid through the one-way valve 49. The suction device 78 may also operate intermittently or in another suitable fashion.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A respiratory mask comprising:
a body defining a breathing cavity, the body having a rim defining an opening to the breathing cavity, the body being shaped and configured to receive a nose and a mouth of a patient through the opening into the breathing cavity, the body defining a positive pressure aperture and a negative pressure aperture which extend into the breathing cavity opposite the opening, the negative pressure aperture being positioned between the positive pressure aperture and a bottom end of the body;
a positive pressure tube coupled to the body, a first end of the positive pressure tube being in fluid communication with the positive pressure aperture, a second end of the positive pressure tube being configured to be in fluid communication with a positive pressure source to receive a flow of gas into the breathing cavity via the positive pressure tube; and
a negative pressure tube coupled to the body, a first end of the negative pressure tube being in fluid communication with the negative pressure aperture, a second end of the negative pressure tube being configured to be in fluid communication with a negative pressure source to facilitate fluid flowing out of the breathing cavity via the negative pressure tube.

2. The mask of claim 1, further comprising a one-way valve 49 mounted to the body in the negative pressure aperture, the one-way valve 49 being configured to permit the fluid to flow only out of the breathing cavity through the negative pressure aperture.

3. The mask of claim 1, wherein the positive pressure tube includes a first tube segment and a second tube segment, the first tube segment extending between the first end of the positive pressure tube and the second tube segment, the second tube segment extending between the first tube segment and the second end of the positive pressure tube.

4. The mask of claim 3, wherein the first tube segment has an L-shape.

5. The mask of claim 4, wherein the positive pressure tube includes a segment swivel connector configured to allow the second tube segment to rotate with respect to the first tube segment, the segment swivel connector including a segment annular flange coupled to the second tube segment and a segment flange receiver coupled to the first tube segment, the segment annular flange being seated in a groove of the segment flange receiver.

6. The mask of claim 1, wherein the negative pressure tube having an L-shape.

7. The mask of claim 1, further comprising a positive pressure swivel connector 50 coupling the positive pressure tube to the body to facilitate rotating the positive pressure tube relative to the body, the positive pressure swivel connector 50 comprising a positive pressure flange 52 attached to the positive pressure tube and a positive pressure receiver 54 attached to the body, the positive pressure flange 52 being seated in a groove defined by the positive pressure receiver 54.

8. The mask of claim 1, further comprising a negative pressure swivel connector 56 coupling the negative pressure tube to the body to facilitate rotating the negative pressure tube relative to the body, the negative pressure swivel connector 56 comprising a negative pressure flange 58 attached to the negative pressure tube and a negative pressure receiver 60 attached to the body, the negative pressure flange 58 being seated in a groove defined by the negative pressure receiver 60.

9. The mask of claim 1, further comprising a sealing member 62 coupled to the rim of the body, the sealing member 62 comprising a malleable material such that the sealing member 62 is configured to form a seal between the body and a face of the patient.

10. The mask of claim 1, further comprising an attachment harness 64 configured to attach the body to a head of the patient.

11. The mask of claim 10, wherein the attachment harness 64 comprises:
a pair of loops 66 mounted to the body, the loops 66 being laterally spaced from each other;
a forehead brace 68 mounted to a top end of the body and configured to abut a forehead of the patient; and
a webbing 74 attached to each one of the pair of loops 66 and to the forehead brace 68, the webbing 74 configured to extend behind the head of the patient.

12. A respiratory mask comprising:
a body defining a breathing cavity, the body having a rim defining an opening to the breathing cavity, the body being shaped and configured to receive a nose and a mouth of a patient through the opening into the breathing cavity, the body defining a positive pressure aperture and a negative pressure aperture which extend into the breathing cavity opposite the opening, the negative pressure aperture being positioned between the positive pressure aperture and a bottom end of the body;
a positive pressure tube coupled to the body, a first end of the positive pressure tube being in fluid communication with the positive pressure aperture, a second end of the positive pressure tube being configured to be in fluid communication with a positive pressure source to receive a flow of gas into the breathing cavity via the positive pressure tube, the positive pressure tube including a first tube segment and a second tube segment, the first tube segment extending between the first end of the positive pressure tube and the second tube segment, the second tube segment extending between the first tube segment and the second end of the positive pressure tube, the first tube segment having an L-shape, the positive pressure tube including a segment swivel connector configured to allow the second tube segment to rotate with respect to the first tube segment, the segment swivel connector including a segment annular flange coupled to the second tube segment and a segment flange receiver coupled to the first tube segment, the segment annular flange being seated in a groove of the segment flange receiver;
a negative pressure tube coupled to the body, a first end of the negative pressure tube being in fluid communication with the negative pressure aperture, a second end of the negative pressure tube being configured to be in fluid communication with a negative pressure source to facilitate fluid flowing out of the breathing cavity via the negative pressure tube, the negative pressure tube having an L-shape;
a one-way valve 49 mounted to the body in the negative pressure aperture, the one-way valve 49 being configured to permit the fluid to flow only out of the breathing cavity through the negative pressure aperture;
a positive pressure swivel connector 50 coupling the positive pressure tube to the body to facilitate rotating the positive pressure tube relative to the body, the positive pressure swivel connector 50 comprising a positive pressure flange 52 attached to the positive pressure tube and a positive pressure receiver 54 attached to the body, the positive pressure flange 52 being seated in a groove defined by the positive pressure receiver 54;
a negative pressure swivel connector 56 coupling the negative pressure tube to the body to facilitate rotating the negative pressure tube relative to the body, the negative pressure swivel connector 56 comprising a negative pressure flange 58 attached to the negative pressure tube and a negative pressure receiver 60 attached to the body, the negative pressure flange 58 being seated in a groove defined by the negative pressure receiver 60;
a sealing member 62 coupled to the rim of the body, the sealing member 62 comprising a malleable material such that the sealing member 62 is configured to form a seal between the body and a face of the patient; and
an attachment harness 64 configured to attach the body to a head of the patient, the attachment harness 64 comprising:
a pair of loops 66 mounted to the body, the loops 66 being laterally spaced from each other;
a forehead brace 68 mounted to a top end of the body and configured to abut a forehead of the patient; and
a webbing 74 attached to each one of the pair of loops 66 and to the forehead brace 68, the webbing 74 configured to extend behind the head of the patient.

13. A respiratory system comprising:
a pressure generation device 76 configured to cause gas flow away from the pressure generation device 76 via positive pressure;
a mask body defining a breathing cavity which is in fluid communication with the pressure generation device 76 via a positive pressure aperture in the mask body, the mask body having a rim defining an opening to the breathing cavity opposite the positive pressure aperture, the mask body being shaped and configured to receive a nose and a mouth of a patient through the opening into the breathing cavity, the mask body defining a negative pressure aperture which extends into the breathing cavity opposite the opening, the negative pressure aperture being positioned between the positive pressure aperture and a bottom end of the mask body; and
a suction device 78 coupled to and in fluid communication with the negative pressure aperture, the suction device 78 including a chamber 80 for collecting fluid, the suction device 78 being configured to apply negative pressure to draw fluids from the breathing cavity of the mask into the chamber 80.

14. The system of claim 13, further comprising a one-way valve 49 mounted to the mask body in the negative pressure aperture, the one-way valve 49 being configured to permit the fluid to flow only out of the breathing cavity through the negative pressure aperture.

15. The system of claim 13, further comprising a positive pressure tube coupled to and extending between the mask body and the pressure generation device 76, the positive pressure tube being in fluid communication with the positive pressure aperture, the positive pressure tube being configured to direct a flow of gas from the pressure generation device 76 to the breathing cavity of the mask body.

16. The system of claim 13, further comprising a negative pressure tube coupled to and extending between the mask body and the suction device 78, the negative pressure tube being in fluid communication with the negative pressure aperture, the negative pressure tube being configured to direct fluid from the breathing cavity to the chamber 80 of the suction device 78.
